(19) United States
(12) Patent Application Publication
(10) Pub. No.: US 2025/0207120 A1
(43) Pub. Date: Jun. 26, 2025

(54) POLYVINYLALCOHOL CARRIER ON WHICH DENITRIFYING MICROORGANISM AND BIOFILM-FORMING MICROORGANISM ARE IMMOBILIZED, METHOD OF REMOVING OR REDUCING NITRIC OXIDE IN SAMPLE BY USING THE SAME, AND METHOD OF PREPARING THE SAME

(71) Applicant: Samsung Electronics Co., Ltd., Suwon-si (KR)

(72) Inventors: Seung Hoon Song, Suwon-si (KR); Woo Yong Shim, Suwon-si (KR); Jae-Young Kim, Suwon-si (KR); Jinha Kim, Suwon-si (KR); Yu Kyung Jung, Suwon-si (KR)

(21) Appl. No.: 18/937,353

(22) Filed: Nov. 5, 2024

(30) Foreign Application Priority Data
Dec. 26, 2023 (KR) 10-2023-0191853

(51) Int. Cl. C12N 11/084; C12P 3/00

(52) U.S. Cl. CPC C12N 11/084; C12P 3/00

(57) ABSTRACT

A method of preparing a carrier on which a denitrifying microorganism and a biofilm-forming microorganism are immobilized, the method including: mixing a polyvinylalcohol, a denitrifying microorganism, and a biofilm-forming microorganism to prepare a mixture; and adding the mixture to a solution containing a crosslinking agent to prepare the carrier on which a denitrifying microorganism and a biofilm-forming microorganism are immobilized. Also, a polyvinylalcohol carrier on which a denitrifying microorganism and a biofilm-forming microorganism are immobilized, and a method of removing or reducing nitric oxide in a sample by using the same.

### CROSS-REFERENCE TO RELATED APPLICATION

This application is based on and claims priority under 35 U.S.C. § 119, and all of the benefits accruing therefrom, to Korean Patent Application No. 10-2023-0191853, filed on Dec. 26, 2023, in the Korean Intellectual Property Office, the content of which is incorporated by reference herein in its entirety.

### BACKGROUND

### 1. Field

The present disclosure relates to a polyvinylalcohol carrier on which a denitrifying microorganism and a biofilm-forming microorganism are immobilized, a method of removing or reducing nitric oxide in a sample by using the same, and a method of preparing the same.

### 2. Description of the Related Art

Nitric oxide (NO) is one of the air pollutants mainly released during fuel combustion. Examples of various nitrogen oxides (NOx) include N₂O, NO, N₂O₃, NO₂, N₂O₄, N₂O₅, and the like. Among these NOx, NO and NO₂ are the main cause of air pollution. N₂O along with carbon dioxide (CO₂), methane (CH₄) and freon gases (e.g. chlorofluorocarbons (CFCs)) are the main cause of the greenhouse effect by which heat is absorbed and stored. Nitrogen oxide is one of the six greenhouse gases regulated under the Kyoto Protocol and has a Global Warming Potential (GWP) value of 310, exhibiting a high warming effect per unit mass compared to CO₂ and CH₄. Additionally, nitrogen oxide is a leading cause of smog and acid rain; it forms second-generation ultrafine dust through chemical reactions in the air, and adversely affects respiratory health by increasing the ground-level ozone concentration.

Nitrogen oxide-converting microorganisms in biological processes reduce NOx to N₂ through a dissimilatory reductive process. The reduction may include, for example, reducing NOx to N₂O as an intermediate, and then reducing the intermediate to N₂.

Biological processes using microorganisms are identified based on methods for supplying nutrients, usually in the form of a sugar, and can include various reaction processes such as batch, fed-batch, and continuous processes. In any of these processes, the microorganisms are immobilized on a reaction carrier for long-term operation and microbial reuse. A variety of methods for immobilizing a microorganism on a carrier may be used, such as absorption, cross-linking, entrapment, and encapsulation, to name a few. However, due to degradation of the carrier resulting in release of the microorganisms, these immobilization methods present environmental concerns.

Therefore, there is a need for a reaction carrier which can maintain its carrier activity for a long period of time even after repeated use, thereby reducing microbial loss, e.g. loss of a denitrifying microorganism, and minimizing contamination from microorganisms released in final discharge water.

### SUMMARY

Provided in an aspect is a method of preparing a carrier on which a denitrifying microorganism and a biofilm-forming microorganism are immobilized, the method including mixing a polyvinylalcohol, a denitrifying microorganism, and a biofilm-forming microorganism, to prepare a mixture; and contacting the mixture and a solution containing a crosslinking agent to prepare the carrier.

Provided in an aspect is a polyvinylalcohol carrier on which a denitrifying microorganism and a biofilm-forming microorganism are immobilized, wherein the denitrifying microorganism is Paracoccus versutus, and the biofilm-forming microorganism is Pseudomonas stutzeri.

Provided in an aspect is a method of reducing an amount of nitric oxide in a sample containing nitric oxide, the method including contacting the sample with a polyvinylalcohol carrier, on which a denitrifying microorganism and a biofilm-forming microorganism are immobilized, to reduce the nitric oxide to molecular nitrogen or an intermediate thereof.

Additional aspects will be set forth in part in the description which follows and, in part, will be apparent from the description, or may be learned by practice of the presented embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram showing prepared polyvinylalcohol carriers on which Paracoccus versutus and Pseudomonas stutzeri are immobilized;

FIG. 2A is a diagram showing results of measuring a degree of microbial loss (A), after a polyvinylalcohol carrier on which P. versutus and P. stutzeri are immobilized is incubated in the presence of FeEDTA-NO;

FIG. 2B is a diagram showing results of measuring a degree of decomposition of nitric oxide after a polyvinylalcohol carrier on which P. versutus and P. stutzeri are immobilized is incubated in the presence of FeEDTA-NO;

FIG. 3 is a diagram showing results of measuring a degree of microbial loss after a polyvinylalcohol carrier on which P. versutus and P. stutzeri are immobilized is incubated repeatedly in the presence of FeEDTA-NO; and

FIG. 4 is a diagram showing results of measuring a rate of decomposition of nitric oxide after a polyvinylalcohol carrier on which P. versutus and P. stutzeri are immobilized is incubated in the presence of FeEDTA-NO.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise. “Or” means “and/or.” “At least one” is not to be construed as limiting “a” or “an.” As used herein, the term “and/or” includes any and all combinations of one or more of the associated listed items. As used herein, the term “or” and “and/or” includes any and all combinations of one or more of the associated listed items. Expressions such as “at least one of,” when preceding a list of elements, modify the entire list of elements and do not modify the individual elements of the list.

It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, ±10% or ±5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

According to an aspect of the disclosure provided herein is a method of preparing a carrier on which a denitrifying microorganism and a biofilm-forming microorganism are immobilized, the method including mixing a polyvinylalcohol, a denitrifying microorganism, and a biofilm-forming microorganism, to prepare a mixture; and preparing carrier particles by contacting the mixture, or adding the mixture, to a solution containing a crosslinking agent.

In some aspects, the denitrifying microorganism may be a microorganism having activity of reducing nitric oxide (NO) to molecular nitrogen or an intermediate thereof. The denitrifying microorganism may be a microorganism of the genus Paracoccus, the genus Pseudomonas, or the genus Klebsiella, or a combination thereof. For example, the denitrifying microorganism may include Paracoccus versutus, Paracoccus denitrificans, Pseudomonas putida, Pseudomonas denitrificans, Pseudomonas stutzeri, or Klebsiella pneumonia. The denitrifying microorganism may be a native strain as well as a recombinant microorganism with a denitrifying activity. The denitrifying microorganism may be P. versutus. The denitrifying microorganism may be artificially cultured. The culture may not contain any microorganism other than the denitrifying microorganism.

In other aspects, the biofilm-forming microorganism may refer to a microorganism that forms a film on a carrier surface. The biofilm-forming microorganism may form a biofilm on a carrier surface, thereby reducing breaking away and loss of the denitrifying bacteria from the carrier during a reaction or reducing loss of activity of the denitrifying bacteria, or a combination thereof. The biofilm-forming microorganism may be a microorganism of the genus Pseudomonas, the genus Bacillus, the genus Azospirillum, the genus Staphylococcus, or the genus Escherichia, or a combination thereof. The biofilm-forming microorganism may be a microorganism of the genus Pseudomonas. The biofilm-forming microorganism may be P. stutzeri. The biofilm-forming microorganism may be artificially cultured. The culture may not contain any microorganism other than the biofilm-forming microorganism. In some aspects, the denitrifying microorganism and the biofilm-forming microorganism are not the same. In other aspects, the biofilm-forming microorganism may have denitrifying activity as well as biofilm-forming activity. The biofilm-forming microorganism may proliferate by co-culture with the denitrifying microorganism, optionally without inhibiting the viability, growth or denitrifying activity of the denitrifying microorganism.

In one aspect, the preparing of the mixture may include preparing a solution containing polyvinylalcohol dissolved by heating a polyvinylalcohol-containing solution. The polyvinylalcohol may have a polymerization degree in a range of about 146,000 to about 186,000. The polyvinylalcohol may be a linear polymer of vinylalcohol represented by [CH₂CHOH]ₙ. The heating may be performed at a temperature at which polyvinylalcohol can be dissolved, for example, a temperature in a range of about 60° C. to about 100° C., about 80° C. to about 100° C., or about 60° C. to about 80° C. The solution containing polyvinylalcohol may include about 5% (w/v) to about 15% (w/v) of polyvinylalcohol, for example, about 5% (w/v) to about 10% (w/v) or about 7% (w/v) to about 8% (w/v) of polyvinylalcohol based on a total volume of the polyvinylalcohol-containing solution. The solution containing polyvinylalcohol may further comprise a polysaccharide. The polysaccharide may be water-soluble and derived from a microorganism. The polysaccharide may include xanthan gum. The solution containing polyvinylalcohol may contain about 0.1% (w/v) to about 1.0% (w/v) of xanthan gum, for example, about 0.1% (w/v) to about 0.5% (w/v) or about 0.2% (w/v) to about 0.3% (w/v) of xanthan gum, based on a total volume of the polyvinylalcohol-containing solution. The polysaccharide, such as xanthan gum, may have a role in forming the carrier into a spherical shape and preventing the carriers from sticking each other.

In other aspects, the preparing of the mixture may include adding the denitrifying microorganism and the biofilm-forming microorganism to a liquid medium to prepare a microbial mixture. The denitrifying microorganism and the biofilm-forming microorganism may be mixed at a ratio of about 14:0.1-5, for example, about 14:0.5-2 or about 14:0.5-1.5. In the preparing of the mixture, the denitrifying microorganism and the biofilm-forming microorganism may be mixed at a ratio of about 0.5 parts by weight to about 3.0 parts by weight, about 0.8 parts by weight to about 1.5 parts by weight, about 0.4 parts by weight to about 0.8 parts by weight, or about 1.6 parts by weight to about 3.0 parts by weight, based on 100 parts by weight of the polyvinylalcohol. The liquid medium may contain water, a medium, or a buffer.

In some aspects, for the preparing of the carrier particles, the crosslinking agent may include at least one ion, or at least two ions. The crosslinking agent may be, for example, boric acid, sodium sulfate, or (mono-, di-, tri-)sodium phosphate, or a combination thereof. The solution containing the crosslinking agent may refer to a solution containing boric acid. The solution containing the crosslinking agent may have a saturated concentration of boric acid, for example, about 3% (w/v) to about 15% (w/v), about 5% (w/v) to about 10% (w/v), about 5% (w/v) to about 8% (w/v), about 7% (w/v) to about 10% (w/v), or about 5% (w/v) to about 6% (w/v). The solution containing the crosslinking agent may refer to a solution containing a surfactant.

In some aspects, the surfactant may simultaneously have a hydrophilic group and a lipophilic group, and thus may be adsorbed to the interface in a dilute solution to reduce the surface tension, and in this regard, may play a role in penetration, dispersion, emulsification, foaming, or the like between substances. The surfactant may be a non-ionic surfactant. The non-ionic surfactant may be Tween®. Tween® may be Tween® 20, Tween® 40, Tween® 80, or Tween® 100. The solution containing the crosslinking agent may contain about 0.1% (w/v) to about 0.5% (w/v) or about 0.2% (w/v) to about 0.3% (w/v) of Tween®.

In some aspects, the method may further include adding the polyvinylalcohol carrier to a solution containing boric acid for incubation and maturation. The polyvinylalcohol carrier may be washed with water. The solution containing boric acid may contain about 5% (w/v) to about 10% (w/v) of boric acid. The incubation may be performed for about 2 hours to about 3 hours. The incubation may be performed at room temperature, or for example at about 4° C. to about 40° C.

In some aspects, the method may further include adding the matured carrier to an aqueous solution of sodium phosphate for incubation and second maturation. The matured polyvinylalcohol carrier may be washed with water. The aqueous solution of sodium phosphate may contain about 5% (w/v) to about 7% (w/v) of sodium phosphate. The incubation may be performed for about 2 hours to about 3 hours. The incubation may be performed at room temperature, or at about 4° C. to about 40° C.

The method may further include adding the carrier matured from the second maturation to water for incubation and third maturation. The polyvinylalcohol carrier matured from the second maturation may be washed with water or filtered. The incubation may be performed for about 2 hours to about 3 hours. The incubation may be performed at room temperature or at about 4° C. to about 40° C.

The method may further include adding the carrier matured from the third maturation to a medium for incubation and activation of the microorganisms. The polyvinylalcohol carrier matured from the third maturation may be washed with water. The incubation may be performed for about 12 hours to about 48 hours. The incubation may be performed under growth conditions for the microorganisms. The incubation may be performed at room temperature, or at about 4° C. to about 40° C. The medium may be a microbial growth medium. The medium may be a glucose-containing 2×YT medium.

According to another aspect of the disclosure, provided is a polyvinylalcohol carrier on which a denitrifying microorganism and a biofilm-forming microorganism are immobilized.

In one aspect, the carrier may be prepared by the aforementioned method. In an aspect, the denitrifying microorganism and the biofilm-forming microorganism are the same as described above. The denitrifying microorganism may be a microorganism belonging to the genus Paracoccus. The denitrifying microorganism may be P. versutus. The denitrifying microorganism may be artificially cultured. The culture may not contain any microorganism other than the denitrifying microorganism. Also, the biofilm-forming microorganism may be a microorganism belonging to the genus Pseudomonas. The biofilm-forming microorganism may be P. stutzeri. The biofilm-forming microorganism may be artificially cultured. The culture may not contain any microorganism other than the biofilm-forming microorganism. These microorganisms may be entrapped and immobilized on the carrier.

In one aspect, the denitrifying microorganism may be P. versutus, and the biofilm-forming microorganism may be P. stutzeri.

According to another aspect of the disclosure, a method of reducing an amount of nitric oxide in a sample having nitric oxide, the method including, providing a sample comprising a nitric oxide, contacting a polyvinylalcohol carrier, on which a denitrifying microorganism and a biofilm-forming microorganism are immobilized, with the sample to reduce the nitric oxide to molecular nitrogen or an intermediate thereof.

In some aspects, the carrier may be prepared by the aforementioned method of preparing a carrier on which a denitrifying microorganism and a biofilm-forming microorganism are immobilized.

In another aspect, the contacting may include incubating the polyvinylalcohol carrier on which the denitrifying microorganism and the biofilm-forming microorganism are immobilized in a medium in the presence of the sample having the nitric oxide.

In other aspects, the medium may be a growth medium for at least one of the denitrifying microorganism and/or the biofilm-forming microorganism, or may be a buffer solution containing glucose as a carbon source for denitrification.

In some aspects, the incubation may be performed under anaerobic conditions. The incubation may be performed at room temperature, for example, about 4° C. to about 40° C., about 15° C. to about 40° C., about 25° C. to about 40° C., or about 25° C. to about 30° C. The incubation may be performed for a time sufficient to remove or reduce the nitric oxide in the sample. The incubation may be performed for, for example, about 6 hours to about 1 week, about 1 day to about 5 days, about 1 day to about 3 days, or about 1 day to about 2 days. The incubation may be performed in a batch, fed-batch, or continuous process. The medium may be a glucose-containing 2×YT medium or a buffer solution containing glucose as a carbon source for denitrification.

According to the method of preparing a carrier on which a denitrifying microorganism and a biofilm-forming microorganism are immobilized, the carrier on which the denitrifying microorganism and the biofilm-forming microorganism are immobilized may be efficiently prepared.

In one aspect, the carrier on which the denitrifying microorganism and the biofilm-forming microorganism are immobilized may be used to remove or reduce the nitric oxide in the sample. By the method of reducing an amount of nitric oxide in a sample according to another aspect, the amount of nitric oxide in the sample may be efficiently reduced.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in detail with reference to Examples below. However, these Examples are for illustrative purposes only, and the scope of the present disclosure is not intended to be limited by these Examples.

### Example 1: Carrier on which Denitrifying Bacteria and Biofilm-Forming Bacteria are Immobilized and Denitrification Using the Same

In this example, a carrier on which denitrifying bacteria and biofilm-forming bacteria are immobilized was prepared, and the carrier thus prepared was cultured in the presence of nitric oxide to carry out denitrification. The denitrifying bacteria refer to microorganisms that have activity of reducing nitric oxide (NOx) to molecular nitrogen or an intermediate thereof.

The biofilm-forming bacteria refer to bacteria that produce carbohydrate-type substances called extracellular polymeric substances (EPSs) and form a film-shaped structure with the EPSs sticking to solid surfaces. The biofilm-forming bacteria produce EPSs, which are carbohydrate-type substances constituting a biofilm on the carrier surface, to bind or physically connect the denitrifying bacteria to each other, thereby reducing breaking away and loss of the bacteria from the carrier during denitrification or reducing loss of activity of the bacteria.

The carrier used polyvinylalcohol (PVA) as a base substance, and the denitrifying bacteria was P. versutus (PV), and the biofilm-forming bacteria was P. stutzeri (PS). The PS is also referred to as biofilm-forming P. stutzeri (bfPS).

### (1) Preparation of Carrier

7 g to 8 g (7% to 8% based on a volume of a final reaction mixture of microorganisms) of PVA (molecular weight of 146,000 to 186,000) and 0.2 g to 0.3 g of xanthan gum were added to 50 mL of distilled water, completely dissolved by heating at 120° C. for 15 minutes, and then cooled to about 40° C., so as to prepare an aqueous solution containing PVA and xanthan gum. Here, the xanthan gum may be mixed in an amount of 0.1 parts by weight to 0.4 parts by weight based on 100 parts by weight of the PVA.

Next, PV and bfPS were added at a concentration ratio (OD₆₀₀) of 14:1 (PV:bfPS) to 50 ml of distilled water at room temperature, so as to prepare a microbial mixture. Here, the OD concentration used in the experiment was 7 for the PV and 0.5 for the bfPS. For the PV and the bfPS, pure-cultured microorganisms were used respectively. For the PV and bfPS, each microorganism was previously pure-cultured by stirring in a 500 ml flask at 30° C. for 24 hrs. The flask contained 200 ml of sterilized culture medium comprising glucose and 2×YT in distilled water. Therefore, each of the PV culture broth and the bfPS culture broth added to the PVA solution did not contain any bacteria other than the PV or the bfPS.

The PVA aqueous solution and the microbial mixture were mixed at room temperature in a 125 ml flask, so as to prepare a mixture of the microorganisms and the PVA. A resulting mixture was then added dropwise to 200 ml of a crosslinking solution (about pH 5), through a thin tube using a peristaltic pump, and then the mixed solution was stirred for 2 hours to 4 hours (2 hours in actual) at room temperature. The crosslinking solution containing 0.2% to 0.4% (0.3% in actual) of Tween® 20 and 5% (w/v) to 10% (w/v) (7% (w/v) in actual) of saturated boric acid in distilled water was used. As a result, spherical carriers on which the PV and the bfPS were immobilized were prepared. This process is called a first maturation process.

Next, the carriers matured in the first maturation process were separated by filtration, and the filtrate was added to a 5% to 7% aqueous solution of sodium phosphate. The mixed solution was then stirred at room temperature for 2 hours to 3 hours for incubation. This process is called a second maturation process.

Next, the reaction products from the second maturation process were separated by filtration. The carriers matured in the second maturation process were added to distilled water, and the mixed solution was stirred at room temperature for 2 hours for incubation. This process is called a third maturation process.

Next, the reaction products from the third maturation process were washed with distilled water. A volume of 100 ml of the washed carriers on which the microorganisms were immobilized was transferred to a 500 ml flask containing 200 ml of culture medium supplemented with glucose (5 g/L) and 2×YT (16 g/L of tryptone, 10 g/L of yeast extract, and 5.0 g/L of NaCl), and then anaerobically cultured at 30° C. for 24 hours at 200 rpm.

The carriers on which the microorganisms were immobilized were separated by filtration from the culture, and then washed three times with distilled water to finally prepare carriers on which the microorganisms were immobilized. This process is also called a reactivation process.

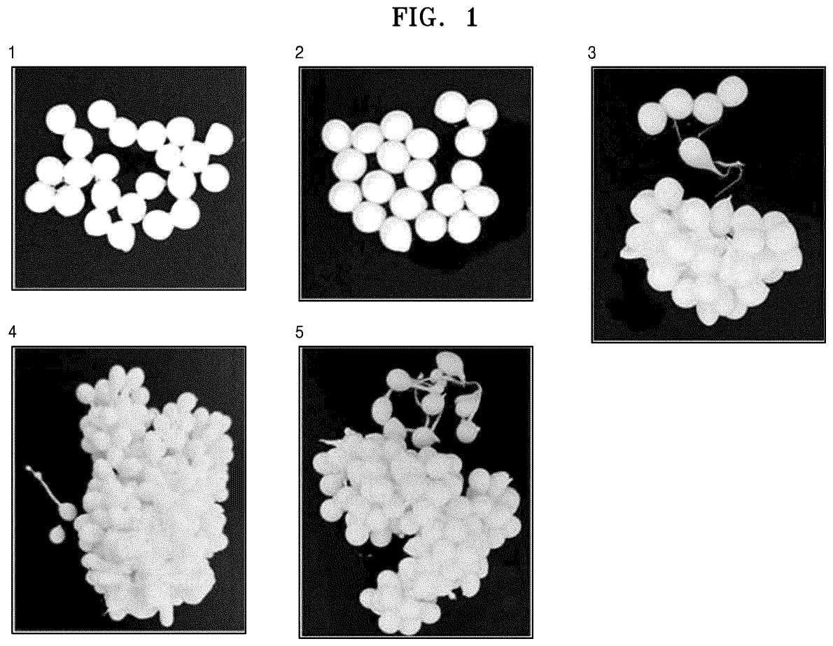

FIG. 1 is a diagram showing the prepared PVA carriers on which P. versutus and P. stutzeri are immobilized. In FIG. 1, Sample Nos. 1, 2, 3, 4, and 5 were prepared by mixing 50 ml of the PVA-containing mixture with 50 ml of a microbial mixture in which PV and bfPS were mixed at a ratio of 15:0, 14:1, 12.5:2.5, 10:5, and 7.5:7.5, respectively, during the preparation of carriers. Sample No. 1 represents carriers of a negative control group not including the bfPS and being immobilized only with the PV.

As shown in FIG. 1, the carriers of a negative control group (Carrier 1), which did not include the bfPS and were immobilized only with the PV, were spherical in shape. Meanwhile, in Sample Nos. 2, 3, 4, and 5, which are carriers of experimental groups including the PV and the bfPS, the carrier shape was changed from spherical to tail, and the amount of agglomerated lumps increased as the proportion of the bfPS increased. In detail, in Carriers 1, 2, 3, 4, and 5, the PV and the bfPS were mixed at a ratio of 15:0, 14:1, 12.5:2.5, 10:5, and 7.5:7.5, respectively. Carrier 2 was a normal spherical carrier, Carrier 3 had some agglomeration during the preparation of carriers, Carrier 4 had some agglomeration and tailing during the preparation of carriers, and Carrier 5 had agglomeration during the preparation of carriers. Such phenomena are believed to be caused by composite substances produced by the bfPS as the proportion of the bfPS increased. Also, the carriers having tails and agglomeration also affected the decomposing activity of nitric oxide. This is because the bfPS itself or composite substances produced therefrom affected the denitrifying activity of the PV or the substance movement of nitric oxide, such as FeEDTA-NO, and reactants, such as gas, and reaction products. However, the claimed invention is not to be understood as being limited to the specific mechanism described herein.

When the carriers of Sample No. 2 were used as a sample in the experimental group, the denitrifying activity of the PV was well maintained and the degree of microbial loss from the carriers was small. Therefore, the ratio of the PV to the bfPS may be 14:0.5 to 2. Also, in the preparation of the carrier, the amount of the microorganisms in the microbial mixture was 1.5 parts by weight to 8 parts by weight based on 100 parts by weight of the PVA.

(2) Denitrification Using Carrier on which Denitrifying Bacteria and Biofilm-Forming Bacteria are Immobilized

The PVA carrier, on which P. versutus as the denitrifying bacteria and P. stutzeri as the biofilm-forming bacteria were immobilized, was brought into contact with nitric oxide (a NOx) to reduce NOx in a sample to molecular nitrogen, thereby reducing the concentration of NOx. For use as the NOx, FeEDTA-NO, in which a dissolved form of NOx is combined with EDTA, was used. FeEDTA-NO was prepared by adding NaNO₂ to a solution containing Fe-EDTA while stirring, wherein pH thereof was adjusted to 7 by using NaOH.

In detail, 50 ml of the carriers was added to a 125 ml flask containing 50 ml of the reaction mixture, and the inlet of the flask was sealed with a silicon stopper to maintain the anaerobic conditions. The flask containing the reaction mixture was then incubated at 30° C. for 14 days at 140 rpm. For the incubation of the reaction mixture, an M9 minimal salts medium) (15 g/L of KH₂PO₄, 2.5 g/L of NaCl, 33.9 g/L of Na₂HPO₄, and 5 g/L of NH₄Cl) supplemented with 5% glucose and 10 mM of FeEDTA-NO was used. The medium was a minimal medium that keeps the immobilized microorganisms in a stationary phase while minimizing the growth of the immobilized microorganisms.

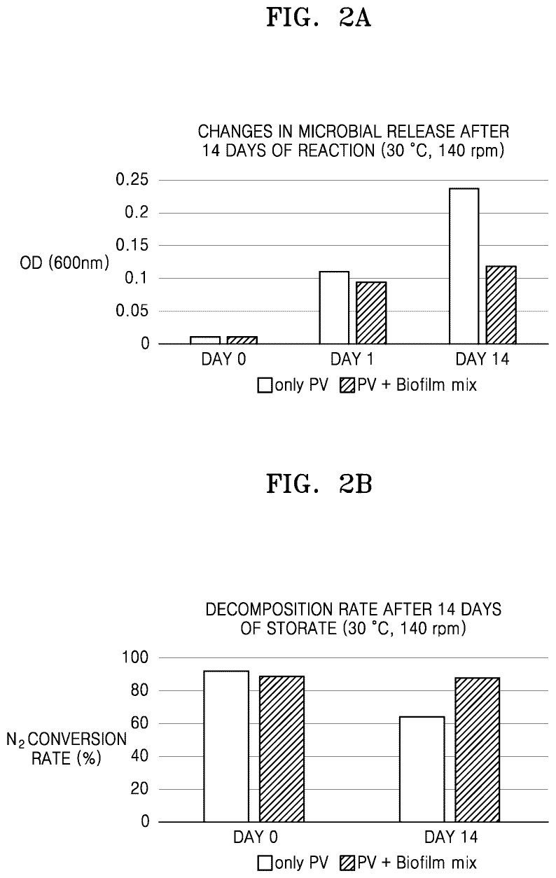

FIGS. 2A and 2B are each a diagram showing the results of measuring a degree of microbial loss (FIG. 2A) and a degree of decomposition of NOx (FIG. 2B), after the PVA carriers on which P. versutus and P. stutzeri were immobilized and were incubated in the presence of FeEDTA-NO. FIG. 2A shows the OD₆₀₀ value measured for the liquid portion of the reaction product excluding the carriers, indicating the degree to which the microorganisms were separated from the carriers and present in the liquid phase, i.e., the degree of release. FIG. 2B shows the results of analyzing the amount of N₂ with a gas chromatography-mass spectroscope (GC-MS) by collecting 100 ul of the gas in the headspace of samples after incubation at different time points, at the beginning of the incubation, i.e., Day 0, and at 14 days after the beginning of the incubation, wherein the samples were incubated under the same conditions for 6 hours in the flask containing FeEDTA-NO. In FIGS. 2A and 2B, “only PV” represents a control group carrier as a carrier immobilized only with PV, and “PV+biofilm” mix represents an experimental group carrier on which PV and biofilm-forming bacteria were immobilized. In the experiments of FIGS. 2A and 2B, the carriers of Sample No. 2 were prepared as described in “(1) Preparation of carrier” above.

As shown in FIG. 2A, as a result of comparing the degree of microbial release from the carriers on Day 1 and Day 14 of the incubation, the carriers without the bfPS had microbial release of about 120% or more compared to Day 1, whereas the carriers including the bfPS had only a 26% increase from Day 1. The results show that, on Day 14 of the incubation, the carriers including the bfPS had the microbial loss of 50% compared to the carriers not including the bfPS (OD value for microbial loss of carrier with bfPS−OD value for microbial loss of carrier without bfPS)/(OD value for microbial loss of carrier without bfPS)×100). Table 1 shows the degree of microbial release on Day 14 corresponding to the results shown in FIG. 2A.

TABLE 1

| Time (day) | Only PV | PV + Biofilm mix |
|---|---|---|
| 0 | 0.01 | 0.01 |
| 1 | 0.109 | 0.094 |
| 14 | 0.236 | 0.118 |

As shown in FIG. 2B, the conversion rate from FeEDTA-NO to N₂, i.e., the nitric oxide decomposition rate, on Day 0 and Day 14 was 92 and 64, respectively, for the control group carriers not including the bfPS, and 89 and 88, respectively, for the experimental group carriers including the bfPS. The results indicate that the experimental group carriers including the bfPS maintained the initial activity even after 14 days, whereas the control group carriers not including the bfPS showed a decrease in the activity of about 30% compared to the initial activity.

The conversion rate was calculated according to the equation below:

Conversion rate (%)=(N₂ production at measurement/maximum N₂ production)×100

The maximum N₂ production refers to the N₂ production when no further N₂ production occurred after 8 hours to 10 hours of the reaction, and the N₂ production at measurement refers to the production measured constantly at 6 hours before the end of the reaction, to compare the conversion rate therewith.

(3) Confirmation of Degree of Microbial Release According to Repeated Use of Carrier on which Denitrifying Bacteria and Biofilm-Forming Bacteria were Immobilized

The degree of microbial release upon repeated use of the carriers was confirmed to confirm the stability of the carrier activity.

50 ml of the carriers was added to a 125 ml flask containing 50 ml of the reaction mixture, and the inlet of the flask was sealed with a silicon stopper to maintain the anaerobic conditions. The flask containing the reaction mixture was then incubated at 30° C. and 140 rpm for 14 days. For the incubation of the reaction mixture, an M9 Minimal salts medium) (15 g/L of KH₂PO₄, 2.5 g/L of NaCl, 33.9 g/L of Na₂HPO₄, and 5 g/L of NH₄Cl) supplemented with 5% glucose and 10 mM of FeEDTA-NO was used. The medium was a minimal medium that keeps the immobilized microorganisms in a stationary phase while minimizing the growth of the immobilized microorganisms. For use as the carriers, the carriers of Sample No. 2 prepared as described in “(1) Preparation of carrier” above were used.

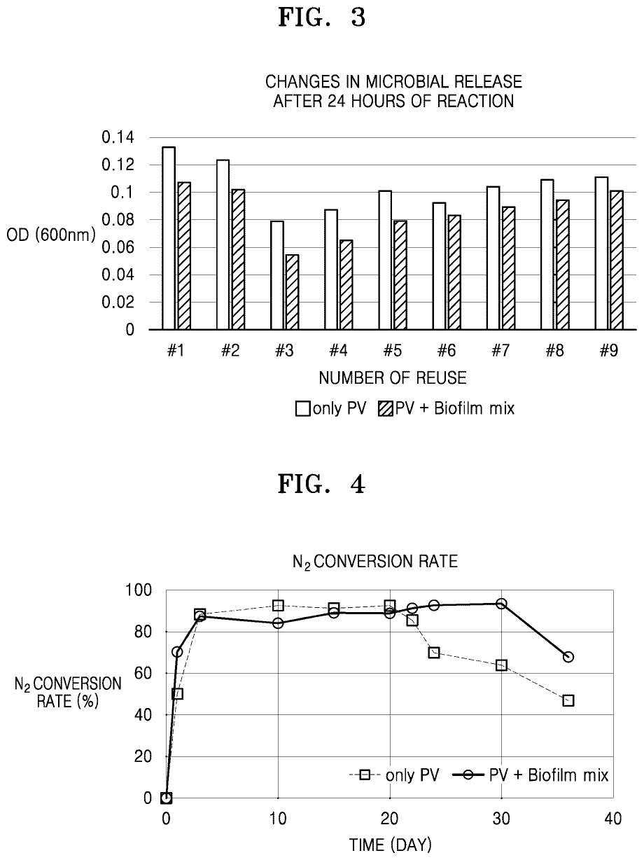

FIG. 3 is a diagram showing the results of measuring the degree of microbial loss after the PVA carriers on which P. versutus and P. stutzeri were immobilized were incubated repeatedly in the presence of FeEDTA-NO.

In FIG. 3, repeatedly used Carrier Nos. 1, 2, 3, 4, 5, 6, 7, 8, and 9 represent the number of times the carriers were used in 24 hours. That is, the results are OD₆₀₀ values measured for the liquid portion of the reaction product excluding the carriers after the flask containing the reaction mixture was incubated at 30° C. for 24 hours at 140 rpm, indicating the degree to which the microorganisms were separated from the carriers and present in the liquid phase, i.e., the degree of release. Here, the carriers used for 24 hours were washed three times using an M9 minimal medium, and FeEDTA-NO was added thereto and incubated in the same manner as the previous recovery.

As shown in FIG. 3, compared to the control group carriers not including the bfPS, the carriers of experimental group including the bfPS showed the microbial release reduced by 10% to 30%. These results indicate that, as the carriers were used repeatedly, the microbial release occurred and affected the stability of the carriers. In FIG. 3, the microbial release decreased in the repeated use 1 to 3, which is believed to be due to the release of the microorganisms immobilized on the outer surface of the carriers. Table 2 shows the data corresponding to FIG. 3.

TABLE 2

| Number of uses | Only PV | PV + Biofilm mix |
|---|---|---|
| #1 | 0.133 | 0.107 |
| #2 | 0.123 | 0.102 |
| #3 | 0.079 | 0.054 |
| #4 | 0.087 | 0.065 |
| #5 | 0.101 | 0.079 |
| #6 | 0.092 | 0.083 |
| #7 | 0.104 | 0.089 |
| #8 | 0.109 | 0.094 |
| #9 | 0.111 | 0.101 |

(4) Confirmation of Carrier Activity According to Repeated Use of Carrier on which Denitrifying Bacteria and Biofilm-Forming Bacteria were Immobilized

The stability of the carrier activity was confirmed based on the conversion rate of NOx to molecular nitrogen when the carriers were used repeatedly.

50 ml of the carriers was added to a 125 ml flask containing 50 ml of the reaction mixture, and the inlet of the flask was sealed with a silicon stopper to maintain the anaerobic conditions. The flask containing the reaction mixture was then incubated at 30° C. for 14 days at 140 rpm. For the incubation of the reaction mixture, an M9 minimal salts medium) (15 g/L of KH₂PO₄, 2.5 g/L of NaCl, 33.9 g/L of Na₂HPO₄, and 5 g/L of NH₄Cl) supplemented with 5% glucose and 10 mM of FeEDTA-NO was used. The medium was a minimal medium that keeps the immobilized microorganisms in a stationary phase while minimizing the growth of the immobilized microorganisms.

The incubation was repeated 9 times over 38 days with intervals of 2 days to 5 days. Here, the carriers used were washed three times using an M9 minimal medium, and FeEDTA-NO was added thereto and incubated in the same manner as the previous incubation.

FIG. 4 is a diagram showing the results of measuring the decomposition rate of nitric oxide after incubating the PVA carriers on which P. versutus and P. stutzeri were immobilized in the presence of FeEDTA-NO. The results were obtained by analyzing the amount of N₂ with a GS-MS by collecting 100 ul of the gas in the headspace after incubation under the same conditions in the flask containing FeEDTA-NO for 6 hours at different timepoints, beginning of the incubation, i.e., Day 0, and on Days 2, 5, 10, 15, 20, 22, 25, 30, and 38. In FIG. 4, only PV represents a control group carrier as a carrier immobilized only with PV, and PV+biofilm mix represents an experimental group carrier on which PV and biofilm-forming bacteria were immobilized.

As shown in FIG. 4, the conversion rate form FeEDTA-NO to N₂, i.e., the nitric oxide decomposition rate, was maintained for up to 30 days in the carriers of experimental group which include the biofilm-forming bacteria when reused multiple times, but was decreased rapidly after 20 days in the carriers of control group which did not include the biofilm-forming bacteria.

The conversion rate was calculated according to the equation below:

Conversion rate (%)=(N₂ production at measurement/maximum N₂ production)×100

The maximum N₂ production refers to the N₂ production when no further N₂ production occurred after 8 hours to 10 hours of the reaction, and the N₂ production at measurement refers to the production measured constantly at 6 hours before the end of the reaction, to compare the conversion rate therewith.

Table 3 shows the data corresponding to FIG. 4.

TABLE 3

| Time (day) | Only PV | PV + Biofilm mix |
|---|---|---|
| 0 | 0 | 0 |
| 1 | 49.8 | 70.5 |
| 3 | 88.4 | 87.3 |
| 10 | 92.6 | 84.1 |
| 15 | 91.2 | 89.2 |
| 20 | 92.4 | 88.8 |
| 22 | 85.4 | 91.4 |
| 24 | 69.8 | 92.7 |
| 30 | 63.9 | 93.3 |
| 36 | 46.9 | 67.8 |

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A method of preparing a carrier on which a denitrifying microorganism and a biofilm-forming microorganism are immobilized, the method comprising:
mixing a polyvinylalcohol, a denitrifying microorganism, and a biofilm-forming microorganism to prepare a mixture; and
contacting the mixture and a solution containing a crosslinking agent to prepare the carrier on which a denitrifying microorganism and a biofilm-forming microorganism are immobilized.

2. The method of claim 1, wherein the denitrifying microorganism is a microorganism of the genus Paracoccus, the genus Pseudomonas, or the genus Klebsiella.

3. The method of claim 1, wherein the biofilm-forming microorganism is a microorganism of the genus Pseudomonas.

4. The method of claim 1, wherein the mixing comprises preparing a solution containing polyvinylalcohol dissolved by heating a polyvinylalcohol-containing solution.

5. The method of claim 1, wherein the mixing comprises contacting the denitrifying microorganism and the biofilm-forming microorganism and a liquid medium to prepare a microbial mixture.

6. The method of claim 5, wherein the denitrifying microorganism and the biofilm-forming microorganism are mixed at a by weight ratio of 14:0.1 to 5.

7. The method of claim 1, wherein, in the mixing, about 0.5 parts by weight to about 3.0 parts by weight of the denitrifying microorganism and the biofilm-forming microorganism, based on 100 parts by weights of the polyvinylalcohol, are mixed.

8. The method of claim 4, wherein the polyvinylalcohol-containing solution contains about 5% (w/v) to about 15% (w/v) of polyvinylalcohol, based on a total volume of the polyvinylalcohol-containing solution.

9. The method of claim 8, wherein the polyvinylalcohol-containing solution further comprises about 0.1% (w/v) to about 1.0% (w/v) of xanthan gum, based on a total volume of the polyvinylalcohol-containing solution.

10. The method of claim 1, wherein the crosslinking agent comprises boric acid.

11. The method of claim 10, wherein the solution containing the crosslinking agent contains about 3% (w/v) to about 15% (w/v) of boric acid, based on a total volume of the solution.

12. The method of claim 1, wherein the solution containing the crosslinking agent comprises a surfactant.

13. The method of claim 12, wherein the surfactant is Tween®.

14. The method of claim 13, wherein the solution containing the crosslinking agent contains about 0.1% (w/v) to about 0.5% (w/v) of Tween®, based on a total volume of the solution.

15. A method of reducing an amount of nitric oxide to molecular nitrogen or an intermediate thereof in a sample containing nitric oxide, the method comprising:
providing a sample comprising a nitric oxide;
contacting the sample with a polyvinylalcohol carrier, on which a denitrifying microorganism and a biofilm-forming microorganism are immobilized, to reduce the nitric oxide to molecular nitrogen or an intermediate thereof in the sample.

16. The method of claim 15, wherein the polyvinylalcohol carrier is prepared by contacting a mixture of polyvinylalcohol, a denitrifying microorganism, and a biofilm-forming microorganism with a solution containing a crosslinking agent.

17. The method of claim 15, wherein the contacting comprises incubating the polyvinylalcohol carrier on which the denitrifying microorganism and the biofilm-forming microorganism are immobilized, in a medium in the presence of the sample including nitric oxide.

18. The method of claim 17, wherein the medium is a growth medium for at least one of the denitrifying microorganism and the biofilm-forming microorganism.

19. The method of claim 18, wherein the incubating is performed under anaerobic conditions.

20. A polyvinylalcohol carrier on which a denitrifying microorganism and a biofilm-forming microorganism are immobilized, wherein the denitrifying microorganism is Paracoccus versutus, and the biofilm-forming microorganism is Pseudomonas stutzeri.
